(19) United States
(12) Patent Application Publication
(10) Pub. No.: US 2009/0053704 A1
(43) Pub. Date: Feb. 26, 2009

(54) STABILIZATION OF NUCLEIC ACIDS ON SOLID SUPPORTS

(76) Inventors: Natalia NOVORADOVSKAYA, San Diego, CA (US); Lee Scott Basehore, Lakeside, CA (US); Jeffrey C. Braman, Carlsbad, CA (US)

Correspondence Address: AGILENT TECHOLOGIES INC, P.O BOX 7599, BLDG E , LEGAL, LOVELAND, CO, 80537-0599, US

(21) Appl. No.: 11/844,578

(22) Filed: Aug. 24, 2007

(51) Int. Cl. C12Q 1/68; C07H 21/04

(52) U.S. Cl. 435/6; 536/22.1; 536/23.1

(57) ABSTRACT

The present invention provides methods, compositions, and kits for the storage and stabilization of biological molecules. The methods comprise applying Tris(2-carboxyethyl)phosphine (TCEP) to at least one biological molecule bound to a solid substrate and storing in an organic solvent. Preferably, the biological molecules are nucleic acids. Compositions and kits for performing the process according to the invention are also provided.

### BACKGROUND OF THE INVENTION

1. Field of the Invention

The present invention relates to the field of storage of biological molecules. More specifically, the present invention pertains to methods, compositions, and kits for stabilizing biological molecules, such as nucleic acids, with a solid substrate.

2. Description of Related Art

Analysis of biological molecules, such as DNA and RNA, is crucial to gene expression studies, not just in basic research, but also in the medical field of diagnostic use. For example, diagnostic tools include those for detecting nucleic acid sequences from minute amounts of cells, tissues, and/or biopsy materials, and for detecting viral nucleic acids in blood or plasma. RNA can be used in expression profiling with microarrays as an indicator of cell response to certain environmental changes, such as addition of a particular pharmaceutical compound. RNA can also be used for cDNA generation, reverse transcription PCR (RT-PCR), and Northern blot analysis, among other methods. The success of any of these techniques is correlated with the quality of the nucleic acid used as a starting material.

The storage of biological molecules without degradation is an important consideration in the practice of molecular biology. After the time and effort exerted into isolating biological molecules, the wrong storage conditions can cause degradation or even destruction of the molecules of interest before they are assayed. Even a minimum amount of degradation can result in poor quality of the biological molecules that are used for subsequent analyses, leading to experimental results that can be inaccurate.

The ease of storage of biological molecules, such as nucleic acids, depends on the type of nucleic acid being stored. For example, DNA molecules are routinely stored in a relatively simple liquid such as water or a Tris-based buffer containing a chelating agent, such as EDTA, either refrigerated or frozen. Unlike DNA molecules, which are relatively stable, RNA molecules are more susceptible to degradation due to the ability of the 2′ hydroxyl groups adjacent to the phosphodiester linkages in RNA to act as intramolecular nucleophiles in both base- and enzyme-catalyzed hydrolysis. Whereas deoxyribonucleases (DNases) require metal ions for activity and therefore can be inactivated by chelating agents, many RNases bypass the need for metal ions by taking advantage of the 2′ hydroxyl group as a reactive species. Indeed, bacterial mRNAs have an extremely short half-life in vivo of only a few minutes. Generally, eukaryotic mRNAs have a longer half-life and are stable for several hours in vivo. However, when cell lysis occurs, eukaryotic mRNAs are no longer in a protected environment and can have a very short lifespan. Isolated RNA is usually stored in RNase-free water or low ionic strength buffer at either −20° C. or −80° C. to avoid degradation by RNases. RNA can also be stored in ethanol as a precipitate at cold temperatures and can be later separated from the ethanol by centrifugation, for example, as a final step in purification.

Tris(2-carboxyethyl)phosphine (TCEP) is a compound that can reduce disulfide bonds to sulfhydryl groups. It has been found to be useful for the stabilization and solubilization of proteins. TCEP has also been proposed by Rhee and Burke as a replacement for dithiothreitol (DTT) in protocols involving nucleic acids (Rhee, S. S., and D. H. Burke, Anal. Biochem. 325:137-143, 2004). These investigators determined that TCEP was more stable than DTT at neutral to basic pH and at elevated temperatures. They also determined that TCEP could stabilize RNA at high temperatures and neutral pH to a greater extent than DTT. In view of these findings, they concluded that TCEP, rather than DTT, could be used as a reductant in nucleic acid and thiophosphate chemistry. However, these investigators did not report any research on the use of reducing agents, such as TCEP, DTT, or β-mercaptoethanol (BME), in the reduction of nuclease activity or the storage of RNA on a wet glass or silica filter.

The current state of the art teaches isolation of nucleic acids based on the adsorption of the nucleic acids on glass or silica in the presence of a chaotropic salt, and subsequent elution from the glass or silica substrate into a buffer or water for storage. As an example, Boom et al. (U.S. Pat. No. 5,234,809) discloses a method for isolating nucleic acids in the presence of a chaotropic substance and then washing with a chaotropic substance-containing solution. A further washing solution composed of alcohol and water followed by the drying of the solid phase-nucleic acid complexes is an optional step in the method of the invention. The chaotropic substance is used for lysis of the cells and binding of the nucleic acids to the substrate. This reference, however, does not discuss the use of chaotropic substances in reduction of nuclease activity. This patent also teaches drying of the nucleic acids bound to the mineral substrate. In fact, in general, the current state of the art teaches quick removal of the nucleic acid from the glass or silica substrate after drying of the nucleic acid-substrate complexes and subsequent storage in water or a low ionic strength buffer at a cold temperature.

In field applications where refrigeration is not available and/or dry ice is not abundant or too costly for shipping the isolated nucleic acid, a method that would allow the storage of nucleic acids at room temperature without degradation of the molecules would be advantageous. It would also be advantageous to have a method that would allow the purification of nucleic acid from a substance, such as blood, on a substrate and the ability to stop the purification with the nucleic acid bound to the substrate. At the convenience of the user or transfer of the nucleic acid-silica complexes to another user, the nucleic acids could be separated from the substrate and assayed. This division of the purification of biological molecules and elution of the biological molecules would allow a user in a hospital, for example, to purify RNA from blood in an automated apparatus and then send the RNA bound to a filter in a stable form to a more specialized laboratory for further processing. The RNA bound to the filter would not have to be sent under frozen conditions, such as packed in dry ice, resulting in significant cost savings and ease in packaging the nucleic acid-silica complexes.

### SUMMARY OF THE INVENTION

The present invention addresses needs in the art by providing methods, compositions, and kits for storing and stabilizing biological molecules from samples, such as cell cultures and blood. The invention is based, at least in part, on the surprising discovery that biological molecules, such as RNA, can be stored at relatively warm temperatures (e.g., above freezing) while bound to a mineral substrate, such a glass fiber filter, without degradation. More specifically, biological molecules that have been treated with a composition comprising a reducing agent, such as TCEP, while bound to a mineral substrate can be stored on the substrate, such as in the presence of an organic solvent, without appreciable degradation. The treatment of the biological molecules with a reducing agent, including the combination of treatment with a reducing agent and storage of the biological molecules bound to the substrate in an organic solvent results in stability of the molecules, especially RNA molecules, at temperatures that are currently considered to be detrimental for stability.

In a first aspect, the invention provides a method of storing and/or stabilizing one or more biological molecules. In general, the method comprises: contacting a biological molecule of interest that is bound to a solid support (also referred to herein as a solid matrix, a solid substrate, or a mineral substrate) with one or more reducing agents; and contacting the biological molecule with one or more organic solvents. In a preferred embodiment, at least one of the reducing agents is TCEP. Exposure of the bound molecule to one or more reducing agents and organic solvent(s) results in stabilization of the biological molecule, and allows for storage of the molecule in a substantially bound state for indefinite periods of time. Optionally, some or all of the reducing agent may be removed from contact with the biological molecule prior to contact with the organic solvent(s). In embodiments, the method comprises storing the bound biological molecule for at least one day at a temperature above freezing, such as at room temperature. For example, the method can comprise: washing biological compounds, such as single-stranded nucleic acids or double-stranded nucleic acids, that are bound to a mineral substrate with a composition comprising a reducing agent; adding an organic solvent to the biological molecule-mineral substrate complexes; and storing the biological molecules bound to the substrate in the organic solvent. In a preferred embodiment, the method can be used to store single-stranded nucleic acids, such as RNA, under conditions that are typically considered unstable for nucleic acids. Optionally, the method can encompass storing the complexes in the organic solvent for an extended period of time at elevated temperatures, such as 37° C.

In another aspect, the invention provides compositions that can be used to stabilize and/or store one or more biological molecules, such as nucleic acids. In general, the compositions comprise one or more reducing agents, such as TCEP, and one or more organic solvents. The composition may also comprise a reducing agent, one or more organic solvents, and a biological molecule of interest, such as a nucleic acid. The composition may comprise a biological molecule adsorbed or otherwise bound to a mineral substrate to form a complex, where the complex has been exposed to one or more reducing agents, and one or more organic solvents. For example, the composition may comprise RNA-glass fiber filter complexes, which have been exposed to TCEP, and 100% ethanol. The compositions preferably comprise one or more biological molecules, such as nucleic acids, proteins, carbohydrates, and/or others. In exemplary embodiments, the compositions comprise stabilized nucleic acids, which have been stabilized by contact with one or more reducing agents and one or more organic solvents, wherein the stabilized nucleic acids are found either in the presence of the reducing agent(s), the organic solvent(s), or both, or have been removed from the reducing agent(s) and/or organic solvent(s).

In an additional aspect, the invention provides kits comprising one or more containers that independently contain a mineral support and a reducing agent. For example, a kit may comprise one or more mineral supports for binding a nucleic acid of interest, one or more organic solvents, one or more reducing agent(s) or a composition comprising one or more reducing agents, one or more wash solutions or buffers, or two or more of these in combination. The kits can be used, for example, to store biological molecules, such as nucleic acids. In preferred embodiments, the kits comprise reagents and supplies for isolating a nucleic acid of interest, stabilizing the nucleic acid, and storing the nucleic acid. Optionally, the kits can contain materials to elute stored biological molecules from the mineral substrate of the kit. In general, the kits comprise materials, reagents, supplies, etc. for use in practicing a method of the present invention.

### BRIEF DESCRIPTION OF THE DRAWINGS

The accompanying drawings, which constitute a part of this specification, illustrate several embodiments of the invention and, together with the written description, serve to explain various principles of the invention. It is to be understood that the drawings are not to be construed as a limitation on the scope or content of the invention.

FIG. 1 depicts quality of Jurkat cell RNA treated with TCEP and stored adsorbed to a glass fiber filter in the presence of ethanol as seen by data from an Agilent 2100 Bioanalyzer.

FIG. 2 demonstrates quality of Jurkat cell RNA treated with additional concentrations and pH of TCEP as seen by data from an Agilent 2100 Bioanalyzer.

FIG. 3 shows the quality of Jurkat cell RNA when treated with varying concentrations of TCEP, pH 5.0, as seen by data from an Agilent 2100 Bioanalyzer.

FIG. 4 shows the effect of Tris in a TCEP-containing buffer on Jurkat RNA stability.

FIG. 5 shows the quality of Jurkat cell RNA when treated with varying concentrations of TCEP, pH 6.0, as seen by data from an Agilent 2100 Bioanalyzer.

FIG. 6 demonstrates the quality of white blood cell RNA treated with TCEP and stored for 3 days at 37° C.

FIG. 7 depicts quality of Jurkat cell RNA when stored dry after treatment with TCEP as seen by data from an Agilent 2100 Bioanalyzer.

FIG. 8 demonstrates quality of Jurkat cell RNA treated with TCEP and stored in 100% ethanol at 37° C. for three days compared to untreated control as seen by data from QRT-PCR using a Stratagene Mx 3000P Real-Time PCR instrument (Panels A and B).

### DETAILED DESCRIPTION OF VARIOUS EMBODIMENTS OF THE INVENTION

Reference will now be made in detail to various exemplary embodiments of the invention. The following description is provided to give details on certain embodiments of the invention, and should not be understood as a limitation on the full scope of the invention.

Broadly speaking, the present invention provides methods, compositions, and kits for storing biological compounds bound to a mineral substrate or filter in the presence of an organic solvent. Accordingly, in one aspect, the invention provides a method of storing and stabilizing biological molecules in the presence of an organic solvent after exposure to a composition comprising one or more reducing agents. In general, the method comprises exposing biological molecules already adsorbed or otherwise bound to a mineral substrate with a composition comprising a reducing agent, adding an organic solvent, and storing for a length of time. In a preferred embodiment, the reducing agent is TCEP. In another preferred embodiment, the composition comprising the reducing agent is separated from the biological molecules adsorbed to the mineral substrate before addition of the organic solvent. The method may comprise the act of adsorbing or binding the biological molecules to the mineral substrate before exposure to the reducing agent. The method may also comprise drying and eluting the biological molecules from the mineral substrate or filter after storage. It is thought that the methods of the invention are most useful for storage at temperatures above 4° C., such as up to 70° C. or more, although the methods will work for storage at temperatures below 4° C. as well.

In a preferred embodiment, the invention provides a method of storing and stabilizing nucleic acids, including single-stranded and double-stranded nucleic acids. The method comprises exposing a sample comprising the nucleic acids bound to at least one mineral substrate (also referred to herein as a mineral support or solid support), to a composition or solution comprising a reducing agent, adding an organic solvent to the mixture, and storing the mixture. In a preferred embodiment, the reducing agent is removed before addition of the organic solvent. The mixture can be stored for an extended length of time without refrigeration and without appreciable degradation of the nucleic acid.

It has been surprisingly discovered that contact of a biological molecule bound to a solid support, such as RNA bound to a glass fiber filter, with a reducing agent, such as TCEP, and an organic solvent (in any order or in combination) results in stabilization of the biological molecule such that it may be stored for an extended period of time without significant degradation, including storage at temperatures that are known in the art to cause rapid and substantial degradation of the biological molecule. The methods of this invention thus allow storage and stabilization of biological molecules that are typically unstable at room temperature or above, such as RNA, from being degraded even under typically harsh conditions, such as 37° C., for extended periods of time, such as at least three days. Current teachings in the art strongly suggest that storage of nucleic acid molecules must occur at cold temperatures (e.g., 4° C. or lower) to avoid degradation of the nucleic acid by nucleases. Therefore, for example, current protocols generally advocate minimizing the amount of time that nucleic acids, especially RNA, are allowed to remain at warmer temperatures. RNA isolation protocols generally suggest keeping the RNA mixture on ice during purification and storing the RNA under as cold a temperature as possible, such as at −80° C. According to the methods of the present invention however, it is possible to store RNA molecules, such as those bound to a solid substrate, for extended periods of time at relatively high temperatures, such as at 37° C. for days, without noticeable loss of integrity of the RNA molecules.

As used herein, the term “biological molecule” refers to any molecule found within a cell or produced by a living organism, including viruses. This may include, but is not limited to, nucleic acids, proteins, carbohydrates, and lipids. In preferred embodiments, a biological molecule refers to a nucleic acid. A biological molecule can be isolated from various samples such as tissues of all kinds, cultured cells, body fluids, whole blood, blood serum, plasma, urine, feces, microorganisms, viruses, plants, and mixtures comprising nucleic acids following enzyme reactions. Examples of tissues include tissue from invertebrates, such as insects and mollusks, vertebrates such as fish, amphibians, reptiles, birds, and mammals such as humans, rats, dogs, cats and mice. Cultured cells can be from prokaryotes such as bacteria, blue-green algae, actinomycetes, and mycoplasma and from eukaryotes such as plants, animals, fungi such as yeast, and protozoa.

In a preferred method of the invention, the biological molecules that are stored are nucleic acids. Any kind of DNA molecule can be stored by this method, such as naturally occurring DNA, for example, genomic DNA, and recombinant DNA such as plasmids, artificial chromosomes, and the like. The size of the DNA is not limited. RNA that can be stored by this method includes mRNA, tRNA, rRNA, and noncoding RNA such as snRNA, snoRNA, miRNA, and siRNA. The size of RNA that can be stored by this method is not limited, but typically ranges from about 20 nucleotides (such as some siRNA) to more than about 5 kb or 6 kb (such as some mRNA).

The mineral substrate used for adsorbing the biological molecule can be any substrate that is capable of binding the molecule of interest. Thus, the “mineral substrate” need not necessarily comprise a mineral. Rather, this term is used herein broadly to describe all solid or insoluble substances to which a biological molecule of interest may bind, be adsorbed, etc. For example, a mineral substrate according to the invention may be polymeric material, such as a membrane, which can be in a single sheet/layer or multiple sheets/layers, made of, for example, polysulfone (PSU; such as BTS membranes from Pall Corp.), polyvinylpyrrolidone (PVP), PSU/PVP composites (e.g., MMM membranes from Pall Corp.), polyvinylidene fluoride (PVDF), nylon, and nitrocellulose. The “mineral substrate” can also comprise composites or combinations of two or more solid/insoluble substrates. For binding of nucleic acids, it is preferably a filter that comprises or consists of porous or non-porous metal oxides or mixed metal oxides, silica gel, sand, diatomaceous earth, materials predominantly consisting of glass, such as unmodified glass particles, powdered glass, quartz, alumina, zeolites, titanium dioxide, and zirconium dioxide. Fiber filters comprised of glass or any other material that can be molded into a fiber filter may be employed in this method. If alkaline earth metals are used in the mineral substrate, they may be bound by ethylenediaminetetraacetic acid (EDTA) or EGTA, and a sarcosinate may be used as a wetting, washing, or dispersing agent. Any of the materials used for the mineral substrate may also be engineered to have magnetic properties. The particle size of the mineral substrate is preferably from 0.1 micrometers (um) to 1000 um, and the pore size is preferably from 2 um to 1000 um. The mineral substrate may be found loose, in filter layers made of glass, quartz, or ceramics, in membranes in which silica gel is arranged, in particles, in fibers, in fabrics of quartz and glass wool, in latex particles, or in frit materials such as polyethylene, polypropylene, and polyvinylidene fluoride. The mineral substrate may be in the form of a solid, such as a powder or it may be in a suspension of solid and liquid when it is combined with a liquid sample. The mineral substrate can be found in layers wherein one or more layers are used together to adsorb the sample. In one embodiment, the mineral substrate is found packed into a spin column or spin cup that can be placed in a microcentrifuge tube. In another embodiment, the mineral substrate is packed into a bigger spin column or spin cup for biological molecule isolation from larger samples. In still another embodiment, the mineral substrate is not packed but is found loose and is mixed with the sample. The mineral substrate can also be found in a filter housing allowing fluids to be passed through by positive air pressure and/or vacuum etc. The methods of the invention can be used for storage of nucleic acids after high-throughput and/or automated purification wherein biological molecules are isolated from many samples. For example, the mineral substrate can be found in a 96-well binding plate.

One or more reducing agents are employed in the method of the present invention. The reducing agent can be any substance that chemically reduces another substance, especially by donating one or more electrons. Specifically, a reducing agent that is a disulfide reductant (i.e., can reduce disulfide bonds) may be particularly appropriate for the method, such as TCEP, BME, and/or DTT. In a preferred embodiment, TCEP, a disulfide reductant with the chemical formula of C₉H₁₅O₆P, is at least one of the reducing agents used in the method. TCEP is also commonly used as TCEP-HCl. For ease of reference, herein, the term TCEP will be used to refer to all forms of the molecule. One can also envision that compounds with substitutions and additions at the carbon atoms of TCEP may also perform as TCEP in terms of allowing biological molecules to be stable at elevated temperatures for extended periods of time. For example, one or more carbons may be substituted with short chain alkyl groups, such as methyl, ethyl, propyl, isopropyl, butyl, pentyl, hexyl, septyl, and octyl. Likewise, hydroxyl substitutions may be permitted at one or more of the carbons, as can carboxyl and carbonyl groups. Nitrogen-containing, sulfur-containing, and oxygen-containing groups may be substituted on one or more carbons as well. However, a modification of TCEP that prevents reduction of disulfide bonds found in nucleases and leads to instability of biological molecules under the conditions discussed may not be as advantageous in the method of this invention. Also, it is established, herein, that (hydroxymethyl)aminomethane (Tris; C₄H₁₁NO₃) or Tris-HCl are different molecules from TCEP (C₉H₁₅O₆P). Tris is also known as Tris base, Tris buffer, tromethamine, tromethane, etc.

According to the methods of storing and/or stabilizing one or more biological molecules, preferably for an extended period of time, the term “storing” means keeping a biological molecule of interest in a substantially unaltered state, such as without it being manipulated to maintain it in its current state. The term “stabilizing” means causing one or more biological molecules to be maintained in a state that does not appreciably change over time. Change can be monitored by any assay relevant to the biological molecule of interest. For example, for nucleic acid molecules, degradation, or lack thereof, can be detected by gel electrophoresis, UV spectrophotometry, PCR assays and/or any other assay that can detect the integrity of the nucleic acid molecules. Not appreciably degraded (or changed) means that the molecule of interest is still intact (not degraded) to the extent needed for analysis of the molecule or use of the molecule for a pre-defined purpose. For example, a molecule that is not appreciably degraded is one in which a collection of such molecules show more than 50% of the molecules to be intact. More preferably, the molecules are more than about 60% intact, such as more than about 70%, 80%, or 90% intact.

According to the present methods, biological molecules, such as nucleic acids, can be stored for extended periods of time without appreciable degradation. In some cases, the biological molecules are stored for an extended period of time at temperatures that are recognized in the art as being incompatible with stable storage of the molecule. For example, in the case of RNA storage, it is generally recognized that the RNA should be stored at a temperature below 0° C., such as at −20° C. or preferably −80° C., to ensure that the RNA remains stable over time. According to the present methods, RNA may be stored at temperatures above 0° C. for amounts of time without appreciable degradation. While not limited to any particular minimum or maximum amount of time, exemplary times for storage include from one minute or less to one hour or more. For example, storage may be performed from about 1 hour to many days, weeks, or even months, such as 12 months. The time that the biological molecule can be stored without degradation depends in part on the molecule of interest and the temperature of storage. Those of skill in the art will recognize that every particular value for minutes, hours, days, months, and years are encompassed by the ranges recited herein, without the need for each particular value to be specifically recited.

The invention provides methods for storing one or more biological molecules. For example, nucleic acids, such as RNA and DNA, can be stored according to these methods for an extended period of time, such as hours to days. The storage can be at frozen temperatures (such as −20° C. or −80° C.), at refrigeration temperatures (such as 4° C.), at room temperatures (such as 20° C. to 24° C.), at elevated temperatures (such as 37° C.), or any temperature in between. Storage of some biological molecules may occur higher than 37° C., such as from about 37° C. to about 60° C. In essence, the temperature for storage is unlimited, but is typically a temperature to which samples being stored or shipped might be exposed. As with times of storage, particular values for temperatures need not be disclosed specifically herein for those of skill in the art to understand that each and every value within the stated ranges is encompassed by this invention.

It is envisioned that storage will primarily occur after the biological molecules of interest are absorbed or bound to a mineral substrate, such as a glass fiber filter. Once the biological molecules of interest are separated from other molecules and are bound to the filter, the biological molecules of interest can be stored until the user wants to manipulate them in biological assays, etc. or ship them to another user. For example, if the biological molecule, such as a nucleic acid, is purified using an automated purification system with glass fiber filters contained within a plastic casing, the plastic casing comprising the purified nucleic acids bound to a glass fiber filter can be shipped at room temperature without the fear of breakage of the plastic due to cold temperatures. There may be instances where this method can also be used to store biological molecules that are already purified. For example, purified RNA molecules may be bound to a glass fiber filter for ease in shipping.

From another viewpoint, the methods of the invention provide ways to stabilize biological molecules on a mineral substrate, such as a glass fiber filter. Biological molecules, such as nucleic acids, can be kept at various temperatures bound to a mineral substrate without degradation of the molecules. It is understood in the art that it is often important to make sure that biological molecules are kept intact during their isolation and storage because the use of degraded molecules in an assay will often lead to inaccurate results. As described above, RNA molecules are unstable at elevated temperatures because of their structure and vulnerability to RNase activity. The methods of the present invention allow biological molecules, such as RNA, to be stored in a stable state for an extended period of time at above refrigeration temperatures.

In embodiments, the methods of the invention comprise contacting biological compound-mineral substrate complexes with one or more reducing agents or a composition comprising one or more reducing agents. At times herein, this contacting is referred to as “washing”. A particular method may also encompass combining the biological compound with a mineral substrate to form a complex, either manually or automatically, before exposure to the reducing agent. For example, the complexes may be formed by adding the biological compound, such as nucleic acids, to a glass fiber filter by hand, under appropriate conditions. The complexes may also be formed by adding the biological compounds to a machine which adsorbs the compounds to a glass fiber filter in an automated fashion. Not only can the formation of the biological compound-mineral substrate complexes be performed manually or automatically, but the methods of any of the steps of the invention can also be done either manually or automatically. For example, the addition of a composition comprising a reducing agent, such as TCEP can be performed by hand or by machine.

The reducing agent may be used in the method as a purified compound or as part of a composition. The composition comprising the reducing agent may be any composition that will allow the biological molecule to remain adsorbed or bound to the filter. Preferably, the composition will comprise salt and organic solvent, such as ethanol, and a reducing agent, such as TCEP. The salts used in these methods may be chaotropic salts, such as guanidinium chloride, guanidinium thiocyanate, guanidinium isothiocyanate, sodium perchlorate, and sodium iodide. Non-chaotropic salts may also be used and include salts of Group I alkali metals, such as sodium chloride, sodium acetate, potassium iodide, lithium chloride, potassium chloride, and rubidium and cesium based salts. As a general matter, any salt that will allow the continued binding of a biological molecule to the mineral substrate in the presence of an organic solvent may be used in this method. The salts in the invention may be one particular salt or may comprise combinations thereof such that a mixture of salts is used. The concentrations of salt in the method can range from about 0 M to 5 M, such as from 1 mM to 500 mM, or from 500 mM to 1 M. Those of skill in the art will recognize that every particular value of salt concentration are encompassed by the ranges recited herein, without the need for each particular value to be specifically recited. The organic solvents applicable at this step comprise ethanol or an organic solvent similar to ethanol as described in detail below, and can range in final concentration from about 25% to about 100%. The concentration of reducing agent, such as TCEP, in the composition can range from about 0.01 mM to about 100 mM. The pH of the composition comprising the reducing agent can be any pH, but will typically range from about 4 to about 8. To maintain the pH in the desired range, one or more buffers may be included in the composition. Those of skill in the art are well aware of the various buffers available for buffering of compositions comprising biological materials. One can envision that a pH above 8 would not be appropriate when the biological molecule is RNA because of the propensity of RNA hydrolysis. However, the pH of the buffer may be above 8 for some other biological molecules. In embodiments, the composition comprising the reducing agent does not comprise (hydroxymethyl)aminomethane (Tris; C₄H₁₁NO₃) or Tris-HCl alone as a distinct molecule.

According to the method, the reducing agent and the biological molecule are caused to come into contact, such as in a composition (e.g., a mixture). In some embodiments, some, essentially all, or all of the reducing agent is removed from the biological molecule-containing composition prior to storage of the biological molecule. Removal may be by physical separation of the reducing agent and biological molecule (e.g., pipetting, decanting, evaporation) by dilution of the reducing agent by large volumes of one or more liquids (e.g., washing or simply raising the volume significantly), or any other means by which the reducing agent can be removed. For example, a reducing agent-containing buffer can be added to a biological molecule adsorbed on a filter, and then can be removed using any suitable technique, including, but not limited to, gravity, centrifugation, positive air pressure, and/or vacuum etc. Methods of separation are well known in the art and therefore will not be described in detail herein. Although not limited to one mode of action, in the case of storage of RNA molecules, this step of the method is thought to reduce or eliminate RNase activity found affiliated with the RNA adsorbed to the filter.

After contact of a biological molecule (such as one bound to a filter) with a reducing agent (e.g., a TCEP-containing buffer), the biological molecule is contacted with an organic solvent or a mixture of two or more solvents. For example, one or more organic solvents can be added to a container containing a biological molecule-filter complex. This step is thought to reduce or eliminate any residual nuclease activity that might remain after the reducing agent treatment. The organic solvent used in the method of the invention can be any organic solvent that allows continued binding of biological molecules to a mineral substrate. The organic solvent can be, but is not limited to, ethanol, acetonitrile, acetone, tetrahydrofuran, 1,3-dioxolane, morpholine, tetraglyme, dimethyl sulfoxide, and sulfolane. Preferably, the organic solvent is ethanol, an organic solvent similar to ethanol, or mixtures thereof. An organic solvent similar to ethanol means a solvent of “like” chemical and physical properties. For example, the solvent may have similar specific gravity, miscibility in water, or other characteristics that allow continued binding of the biological molecule to the mineral substrate or filter. “A mixture thereof” means that more than one kind of organic solvent may be used in the buffer. For example, a mixture of ethanol and dioxolane, a mixture of sulfolane and dioxolane, a mixture of ethanol, dioxolane, and acetonitrile, etc. may be used for continued binding of the biological molecule to the mineral substrate. There are many variations of mixtures of organic solvents that can be used for this step and the mixture may comprise more than two organic solvents. The final concentration of organic solvent may be any amount that allows for the continued binding of the molecule of interest. For nucleic acids, it can range from about 50% to 100%, such as from 70% to 100%, for example from 90% to 100%.

The biological molecules adsorbed to the filter can be stored in the organic solvent for an extended period of time. Depending on the biological molecule of interest, storage can be anywhere from minutes, and more likely, from hours to days. In the example of RNA molecules, the methods of the invention can be used to store RNA for at least three days at 37° C., which is a surprising result because such conditions are widely recognized and taught in the art to be extremely adverse for storage of RNA. In the case of DNA molecules, storage can be for days or months without appreciable degradation. In the case of some proteins that do bind or are adsorbed onto a mineral substrate, stability will depend on the specific protein of interest, but will also be in the range of days to months or more.

As noted above, in embodiments, the method is performed on biological molecules bound to mineral substrates, such as RNA bound to glass fiber filter materials. Where the molecule of interest is bound to a mineral substrate, the methods of the invention can comprise eluting the biological molecules from the mineral substrate after storage, such as after storage in an organic solvent. The step of eluting the biological molecule from the mineral substrate can comprise first drying (e.g, by simple evaporation in air) the mineral substrate to eliminate water and the organic solvent (e.g., ethanol), then adding a liquid, such as elution buffer or water, to the substrate, optionally allowing the liquid to stay in contact with the substrate and molecule of interest for a sufficient amount of time to cause elution, (e.g., from about 5 seconds to one hour or more), and separating the liquid from the substrate. Under some circumstances, prior to elution, the bound biological molecules can be exposed to a highly volatile organic compound, such as acetone, to facilitate removal of water and other organic compounds by evaporation. In embodiments where nucleic acids are being eluted, incubation typically can occur from about one second to about 20 minutes, such as from about zero seconds to about 10 minutes, or from about zero to about 5 minutes. In a preferred embodiment, incubation occurs for about 2 minutes. During this step, most of the nucleic acid molecules bound to the substrate should elute into the liquid. Incubation can occur with a liquid that is warm, such as from about 26° C. to about 80° C. or close to room temperature, such as from about 20° C. to about 25° C. Preferably, where the elution solution (e.g., buffer) comprises salts, the salts have a pKa value from about 6 to about 10 and the buffer has a salt concentration up to about 100 mM. For example, 10 mM Tris (pKa 8.0) pH 8.5 may be used to elute the biological molecule from the mineral substrate.

Thus, in embodiments, the invention provides a method for storage of biological compounds, such as nucleic acids, wherein the method comprises: a) adding a composition comprising a reducing agent to at least one biological molecule bound or adsorbed to a mineral substrate; b) optionally removing the reducing agent from the biological molecule bound to the mineral substrate; c) adding an organic solvent to the biological molecule adsorbed to the mineral substrate; and d) storing the biological molecule adsorbed to the mineral substrate for a period of time. In one exemplary embodiment, the biological molecule of interest is an RNA molecule and the reducing agent is TCEP. The methods may also encompass the act of adhering the biological molecule to the mineral substrate before addition of the reducing agent and/or drying the filter and eluting the biological molecule from the filter after storage.

In another general aspect, compositions that can be used to store and stabilize one or more biological molecules are provided. The composition may comprise a reducing agent and an organic solvent. In a preferred embodiment, the composition comprises TCEP. The composition may also comprise a reducing agent, an organic solvent, and a biological molecule, such as nucleic acid. Compositions comprising a biological molecule-mineral substrate complex that has been exposed to a reducing agent and an organic solvent are provided. In general, a composition of the invention comprises a mineral substrate or filter, an organic solvent and at least one biological molecule, such as a double-stranded nucleic acid (e.g., DNA), a single-stranded nucleic acid (e.g., RNA), or a protein, polypeptide, or peptide. In some embodiments, the compositions comprise a sufficient amount of organic solvent and reducing agent (e.g., TCEP) to allow continued adsorption of the biological molecule to the mineral filter and to allow stabilization of the biological molecule. Various ranges of organic solvent and reducing agent that are useful in the methods of the invention, and thus the compositions of the invention, are disclosed above, and any of those ranges or particular concentrations may be used in a composition of the invention. In addition, various salts and concentrations of salts are discussed in the context of the methods of the invention above. Any of those salts, combinations of salts, ranges, or particular concentrations may be used in a composition of the invention. In addition, the various types and amounts of mineral supports that may be present in the compositions are disclosed herein.

In embodiments, the invention provides stabilized nucleic acids. The stabilized nucleic acids are those that result from a method of stabilization according to the present invention. Thus, for example, the stabilized nucleic acids may be those that have been treated with reducing agent, such as TCEP, and at least one organic solvent. In embodiments, the stabilized nucleic acids are present in a composition comprising at least one organic solvent and, optionally, a reducing agent. In the compositions, the reducing agent may be present at relatively high concentrations (e.g., millimolar ranges) or relatively low concentrations (e.g., micromolar, nanomolar, picomolar ranges). In some instances, the reducing agent is present only to the extent that it was not removed by washing or other actions intended to remove the reducing agent. In some instances, the reducing agent is present as a result of dilution of a composition comprising the reducing agent with one or more organic solvents. In some embodiments, stabilized RNA is provided. In these embodiments, the stabilized RNA is created by contacting the RNA with one or more reducing agents and contacting the RNA with one or more organic solvents. Preferably, the RNA is contacted with the reducing agent(s) prior to contacting with the organic solvent(s). In some instances, some, essentially all, or all of the reducing agent(s) is removed from the RNA prior to contacting the RNA with the organic solvent(s). Optionally, the RNA is bound to a solid support prior to exposure to the reducing agent(s), the organic solvent(s), or both.

In yet another general aspect, the present invention provides kits. In general, the kits comprise packaging for holding one or more containers. Typically, the containers contain at least one reagent, supply, or material for practicing a method of the invention. In preferred embodiments, the kit comprises a reducing agent (e.g., TCEP) and an organic solvent which, when used according to the methods of the invention, stabilizes a biological molecule and allows it to be stored without degradation. In embodiments, the kit comprises one or more containers holding an appropriate amount of reducing agent and organic solvent to stabilize at least one nucleic acid molecule. The kits can comprise other components, such as some or all of the components necessary to practice a method of the invention. For example, the kits may comprise one or more mineral substrates or substrate units (e.g., multiple layers of mineral substrates provided as a single unit). Other non-limiting examples of components that may be included in the kits of the invention are sterile water, cell lysis buffer, wash buffers, and elution buffers or water. Of course, multiple organic solvents may be provided, independently or in mixtures of solvents.

### EXAMPLES

The invention will be further explained by the following Examples, which are intended to be purely exemplary of the invention, and should not be considered as limiting the invention in any way.

### Example 1

### Effect of TCEP on Jurkat RNA Stability on Glass-Fiber Filter

In general, RNA was isolated from a Jurkat cell line or human white blood cells using the following protocol for all the experiments in the Examples. The cells (1×10⁷) were collected on glass-fiber spin cups in 50 ml tubes. The cells were washed with 10 ml and then 5 ml of PBS buffer (GIBCO formulation) to reduce contaminants. The filter was transferred to fresh tubes and 3 ml of Lysis Buffer (5 M guanidine thiocyanate, 20 mM sodium citrate pH 7.0, 0.05% sarcosyl, 1% Triton X-100, 0.01% Anti-foam A, 5 mM TCEP pH 5.0) was passed through the filter resulting in the release of nucleic acids from the cells. Genomic DNA was adsorbed to the glass fiber filter in the lysis step. The filtrate, comprising mainly RNA, was measured, an equal volume of 80% sulfolane was added to the filtrate, and aliquots of the filtrate (about 500 ul) comprising the sulfolane were passed through glass-fiber spin cups in 1.5 ml microcentrifuge tubes. Because of the addition of sulfolane, RNA in the filtrates was adsorbed to the glass-fiber filters in this step. The glass-fiber spin cups were washed with 500 ul of Low Salt Wash Buffer (LSW buffer; 2 mM Tris pH 6-6.5, 20 mM NaCl, 80% ethanol) comprising varying concentrations and pH of TCEP, three times. The spin-cups were centrifuged an additional time to dry the glass-fiber filters. RNA for control samples was eluted in 100 ul water and stored at −20° C. The other spin-cups were transferred to fresh microcentrifuge tubes and 100% ethanol (200 ul), or in some cases, LSW buffer comprising varying concentrations and pH of TCEP, was added to each spin-cup. The tubes were sealed with parafilm and stored at 37° C. or room temperature for three days. After storage, the spin-cups were washed with LSW Buffer once and the RNA was eluted with 100 ul of water. The RNA was stored at −80° C. before the assays were performed. The purity and integrity of the RNA was checked using an Agilent 2100 Bioanalyzer, and in some cases, by PCR assays. The Agilent Bioanalyzer runs mini-gels and shows an electrophoregram image and gel-like image of the sample, and automatically evaluates RNA quality (RIN and 28S/18S ratio).

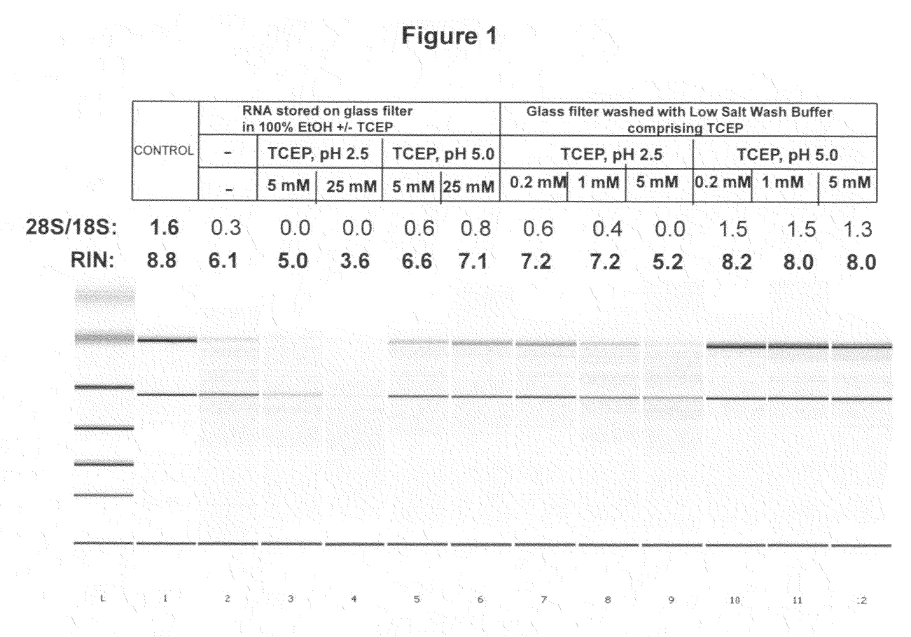

FIG. 1 depicts one set of experiments in which RNA was stored in 100% ethanol and varying concentrations and pH of TCEP and another set of experiments in which RNA was washed with LSW Buffer comprising varying concentrations and pH of TCEP and stored only in 100% ethanol. All samples were stored on glass-fiber filters for 3 days at 37° C. Agilent Bioanalyzer traces demonstrated that the addition of TCEP, pH 5.0, in the first set of experiments, where TCEP was added to the storage composition, slightly increased RNA stability on the glass-fiber filter with respect to RNA Integrity Numbers (RIN) and 28/18 S ribosomal RNA ratios (lanes 5 and 6 compared to the control, lane 2). More specifically, the addition of 5 mM and 25 mM of TCEP, pH 5.0, to the 100% ethanol used for storage, resulted in RIN numbers of 6.6 and 7.1 (lanes 5 and 6), respectively, compared to a RIN number of 6.1 for the sample in which TCEP was not added (lane 2). The 28S/18S ratios of 0.6 and 0.8 for the TCEP, pH 5.0 samples of 5 mM and 25 mM, respectively, were also favorable as compared to a 28S/18S ratio of 0.3 as shown for the sample without TCEP (lane 2). The addition of 5 mM or 25 mM TCEP, pH 5.0, to the storage composition resulted in more 28S and 18S rRNA (lanes 5 and 6) compared to the sample in which TCEP was not added (lane 2). The addition of TCEP, pH 2.5, at either 5 mM or 25 mM to the storage composition was not beneficial for storage of the RNA in this experiment as can be seen by 28S/18S ratios of 0 and the small amount of intact RNA seen by gel electrophoresis (lanes 3 and 4). The control sample in FIG. 1 (lane 1) comprised RNA that was eluted in water and immediately frozen at −20° C. without being stored at 37° C. in the presence of ethanol.

In the second set of experiments in FIG. 1, TCEP was added to the LSW Buffer used for washing instead of to the 100% ethanol composition used for storage of the RNA. Agilent Bioanalyzer traces showed favorable RIN numbers of 8.2, 8.0, and 8.0 for the samples in which TCEP, pH 5.0 was added at concentrations of 0.2 mM, 1 mM, and 5 mM, respectively (lanes 10-12), to the LSW Buffer as compared to a RIN number of 6.1 for the sample without any addition of TCEP (lane 2). Favorable 28S/18S ratios were seen as well of 1.5, 1.5, and 1.3 for the samples in which TCEP, pH 5.0, was added at concentrations of 0.2 mM, 1 mM, and 5 mM, respectively, to the LSW Buffer (lanes 10-12) as compared to a 28S/18S ratio of 0.3 for the sample without any addition of TCEP (lane 2).

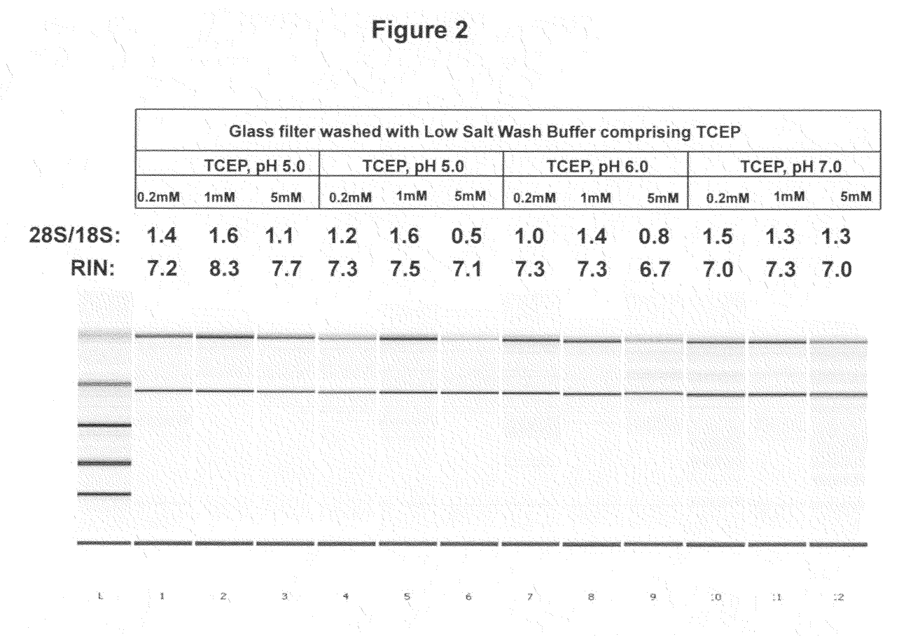

FIG. 2 shows the effect of additional variations in concentrations (0.2, 1, and 5 mM) and pH (5.0, 6.0, and 7.0) of TCEP added to the LSW Buffer. After washing the RNA bound to the glass fiber filter with LSW buffer and removing the buffer by centrifugation, the RNA-glass fiber filter complexes were stored in 100% ethanol for three days at 37° C. The condition in which TCEP, pH 5.0, was added to the buffer was done in duplicate and demonstrates that a 1 mM concentration of TCEP at pH 5.0 in the buffer (lanes 2 and 5) resulted in the best integrity of RNA as seen by Agilent Bioanalyzer traces compared to lanes 1, 3, 4, and 6. More specifically, the RIN numbers seen for the 1 mM TCEP, pH 5.0, samples were 8.3 and 7.5 (lanes 2 and 5, respectively), which were the highest RIN numbers determined in the experiment. The 28S/18S ratios of 1.6 for both 1 mM TCEP, pH 5.0, samples were also the highest ratios seen in the experiment. In fact, the results from FIGS. 1 and 2 show that, in general, RNA bound to a glass fiber filter can be stored in the presence of 100% ethanol for at least three days at 37° C., when the samples are pre-treated or previously washed with LSW buffer comprising 0.2 mM, 1 mM, or 5 mM TCEP at a pH of 5.0, 6.0, or 7.0.

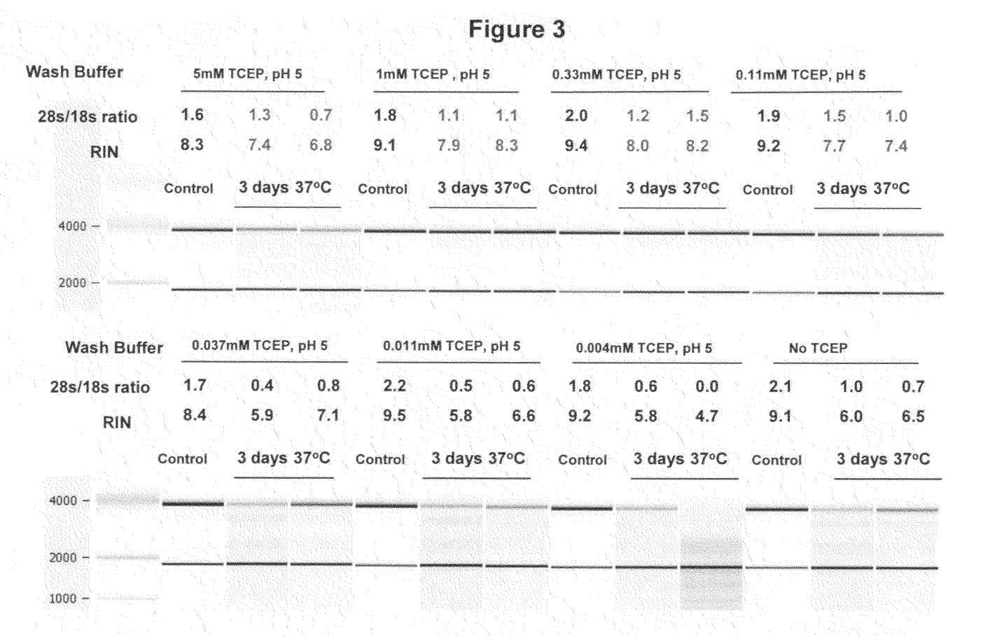

FIG. 3 depicts the effect of still additional variations in concentrations of TCEP (pH 5) added to the LSW (wash) buffer. The RNA-glass fiber filter complexes were stored in 100% ethanol for three days at 37° C. The control samples in FIG. 3 comprised RNA that was eluted in water and immediately frozen at −20° C. without being stored at 37° C. in the presence of 100% ethanol. Results from duplicate samples suggested that TCEP concentrations of 0.33 mM resulted in the best integrity of RNA as seen by Agilent Bioanalyzer traces. Specifically, the RIN numbers seen for the 0.33 mM TCEP samples were 8.0 and 8.2 and the 28S/18S ratios were 1.2 and 1.5. The results from the 1 mM TCEP samples also were favorable as seen by the RIN numbers (7.9 and 8.3) and the 28S/18S ratios (1.1 and 1.1). The figure shows that, under these conditions, a TCEP concentration of 0.1 mM to 5 mM can be advantageously used.

### Example 2

### Effect of Tris on RNA Stability in Wash Buffers Containing TCEP

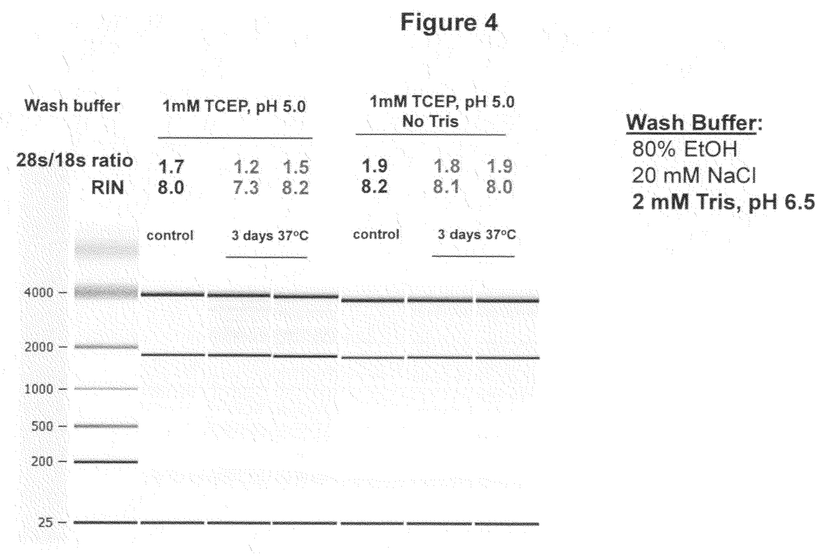

To test the effect of Tris on RNA stability, RNA samples from Jurkat cells were processed essentially as described above, with the exception that, in some cases, Tris was not added to the LSW. Characteristics of the resulting RNA are shown in FIG. 4. In summary, RNA isolated using a wash buffer containing 1 mM TCEP, pH 5.0, without Tris showed improved RNA stability after 3 days at 37° C., as compared to use of a buffer with 2 mM Tris. That is, the Jurkat RNA isolated and stored using TCEP buffer without Tris showed an RIN of 8.1 and 8.0, and a 28S/18S ratio of 1.8 and 1.9. In contrast, Jurkat RNA isolated and stored using TCEP buffer that included Tris at 2 mM showed an RIN number of 7.3 and 8.2 and a 28S/18S ratio of 1.2 and 1.5. Thus, under these conditions, using wash buffer that includes TCEP but lacks Tris can be advantageous.

### Example 3

### Analysis of TCEP Concentration on RNA Stability in the Absence of Tris

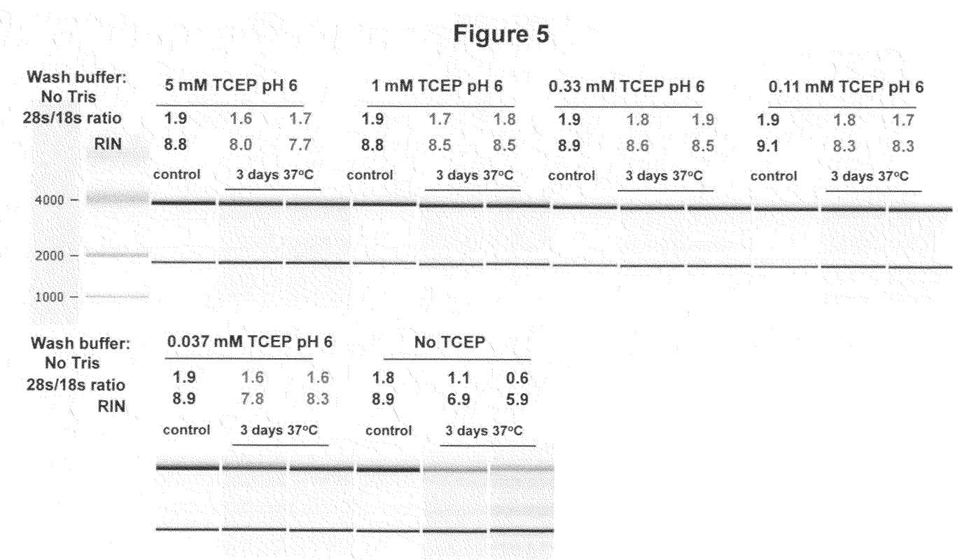

Having established the beneficial effects of TCEP on RNA stability and the deleterious effect of a combination of TCEP and Tris, as compared to TCEP alone, the effect of different concentrations of TCEP on RNA stability, in the absence of Tris, was examined. To do this, RNA from Jurkat cells was isolated as described above, using LSW buffers containing TCEP, pH 6.0, but lacking Tris. The concentration of TCEP in the LSW buffers was varied from 5 mM to 0.037 mM. Buffer lacking both Tris and TCEP was also used. Samples were isolated and stored on glass fiber filters for 3 days at 37° C. The results are shown in FIG. 5. As can be seen from the figure, all samples isolated using buffers containing TCEP, pH 6.0, at the tested ranges showed acceptable stability, whereas samples isolated without TCEP were less stable. The use of anywhere from 0.037 mM TCEP to 5 mM TCEP, pH 6, provided an improvement to RNA stability.

### Example 4

### Effect of TCEP on RNA from White Blood Cells

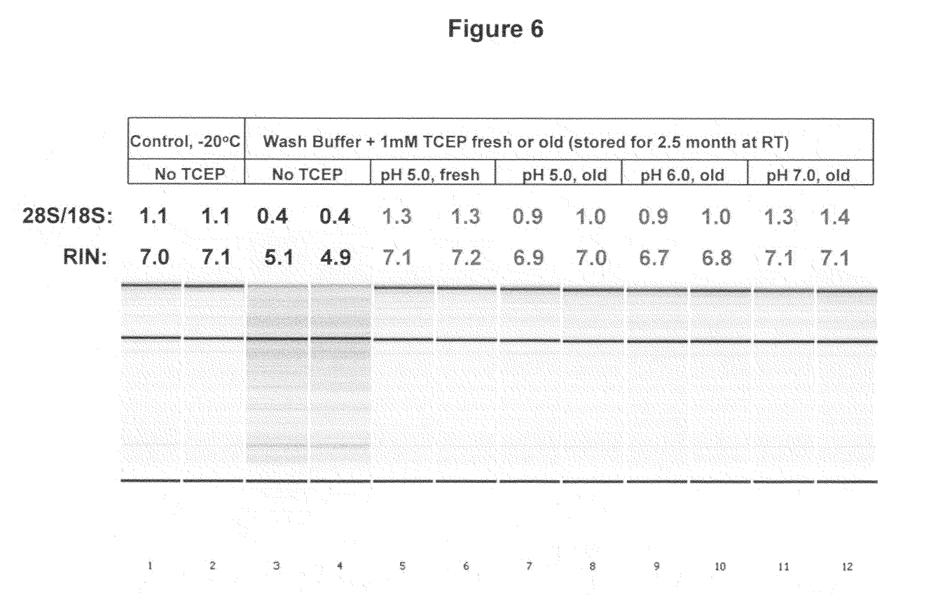

To better characterize the effect of TCEP on RNA stability across cell types, RNA from white blood cells was isolated as described above, using LSW buffer that included 1 mM TCEP at pH 5.0, 6.0, and 7.0. The low salt wash (LSW) buffer with TCEP was freshly made or stored for two and one-half months at room temperature, then used. After washing the RNA was stored on glass fiber filters in 100% ethanol for three days at 37° C. (lanes 5-12). The results were compared to samples isolated in the absence of TCEP (immediately eluted and stored at −20° C. (lanes 1-2) or stored at 37° C. for three days (lanes 3-4). The results are shown in FIG. 6. As can be seen from the figure, white blood cell RNA samples isolated with 1 mM TCEP showed excellent quality when stored for three days at 37° C., whereas RNA samples isolated without TCEP and stored at 37° C. for the same period of time showed significant degradation. These results also demonstrated that LSW buffer with TCEP can be stored at room temperature for at least two and one-half months without losing its activity.

### Example 5

### Effect of TCEP on Jurkat RNA Stability on Glass-Fiber Filter Stored Wet and Dry

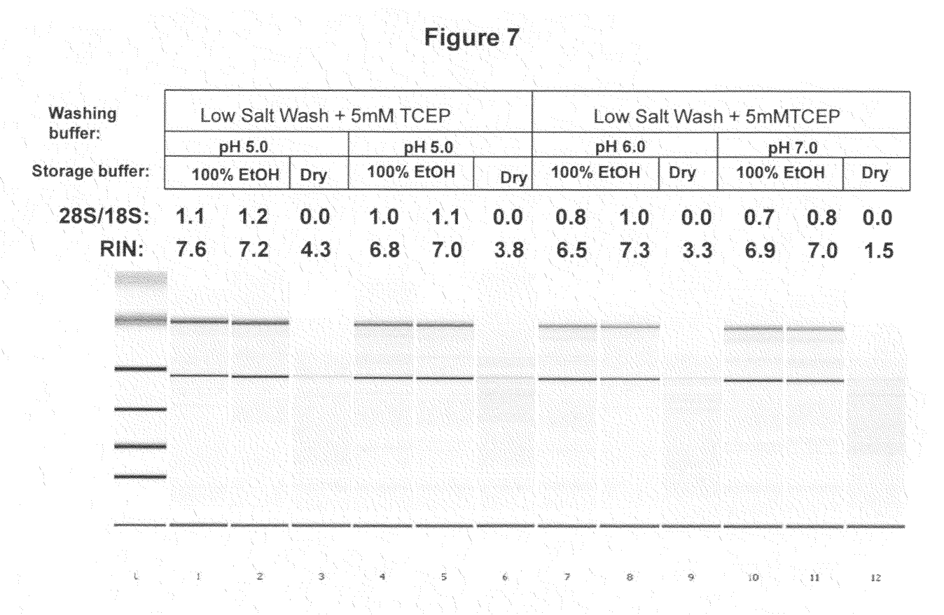

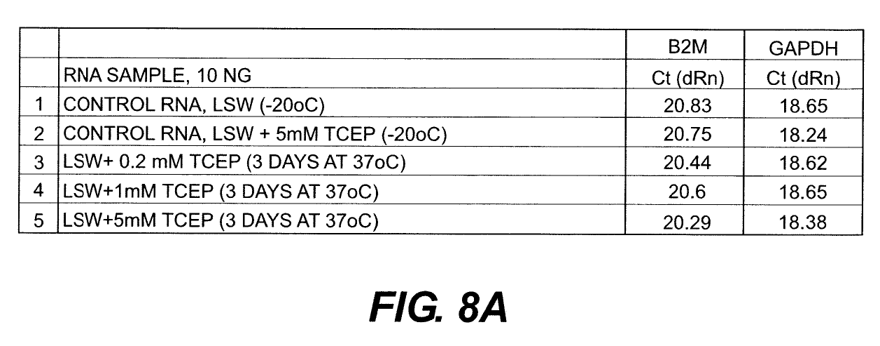

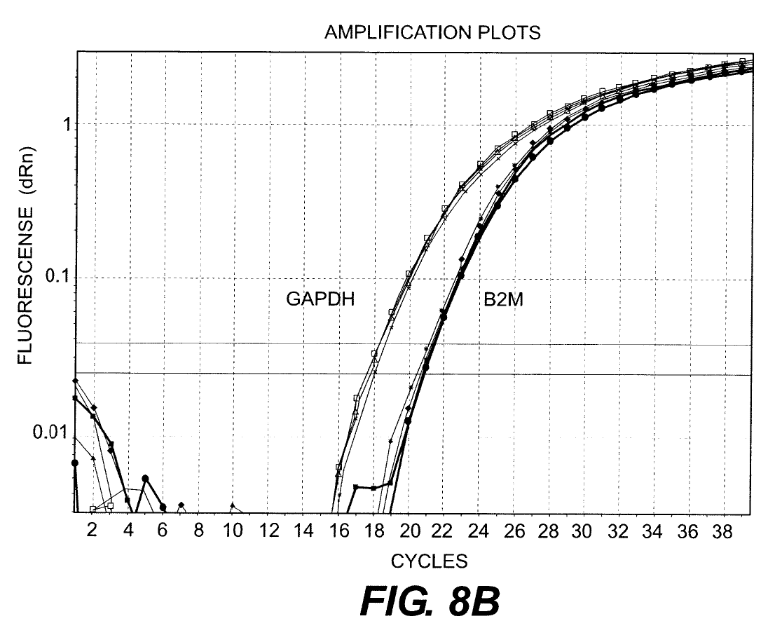

In FIG. 7, RNA was isolated from a Jurkat cell line as described in Example 1. The goal of these experiments was to determine if RNA bound to a glass filter could be stored dry instead of being stored wet in the presence of 100% ethanol. The RNA samples adsorbed to glass filters were washed with LSW buffer comprising 5 mM TCEP at varying pH conditions and stored with or without 100% ethanol for 3 days at 37° C. Results from this experiment showed that washing with LSW buffer comprising 5 mM TCEP at any of the pH values tested (5.0, 6.0, and 7.0) and storing the samples adsorbed to the glass filters in a dry state resulted in little intact RNA, as seen by a low 28S/18S ratio of 0.0 and low RIN numbers. The other samples, which were treated with the same conditions as the dry samples, except that they were stored bound to glass fiber filters in the presence of 100% ethanol, were found to be stable when TCEP at a pH of 5.0, 6.0, or 7.0 was added to the LSW buffer (lanes 1, 2, 4, 5, 7, 8, 10, and 11).

### Example 6

### Evaluation of Jurkat RNA Quality by QRT-PCR

Quantitative Real Time PCR (QRT-PCR) of the purified RNA can be used to show the quality of nucleic acid. In this experiment, the control RNA samples consisted of a sample that was washed with LSW buffer, eluted with water, and stored at −20° C. (sample 1 of Panel A) and a sample that was washed with LSW buffer containing 5 mM TCEP, eluted with water, and stored at −20° C. (sample 2). The rest of the samples were washed with LSW buffer containing 0.2 mM, 1 mM, or 5 mM of TCEP at pH 5.0 (samples 3, 4, and 5, respectively) and stored at 37° C. for three days in 100% ethanol. Evaluation of RNA quality by reverse transcription and amplification of beta-2-microglobulin (B2M) and glyceraldehyde-3-phosphate dehydrogenase (GAPDH) mRNA using QRT-PCR showed equivalent RNA quality for all the samples tested (Panel B). More specifically, FIG. 5 shows amplification plots of Real-time QRT-PCR reactions that were performed using 10 ng of each RNA (25 ul reaction volume), Brilliant QRT-PCR Master Mix, 1-step (Stratagene) and TaqMan primers and probe (B2M and GAPDH, Assay on Demand, ABI) on the Mx3000P Real-time PCR System (Stratagene) using the following cycling parameters: 50°/30 min, then 95°/10 min followed by 40 cycles of 95°/15 sec; 60°/1 min. All five RNA samples showed very similar Cts for two tested genes and perfectly overlapping amplification curves, suggesting that all five tested RNA samples have an equally high quality. Thus, this experiment shows that the addition of up to 5 mM TCEP at pH 5.0 in the LSW buffer not only results in stable RNA samples under these conditions, but also does not affect or inhibit the QRT-PCR reaction.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. A method for stabilizing a biological molecule bound to a solid substrate, said method comprising:
contacting the biological molecule with at least one reducing agent; and
contacting the biological molecule with at least one organic solvents,
wherein said method steps results in stabilization of the biological molecule bound to the solid substrate.

2. The method of claim 1, further comprising removing some or all of the reducing agent(s).

3. The method of claim 1, further comprising storing the biological molecule in the presence of the organic solvent.

4. The method of claim 3, wherein the biological molecule is stored for period of time between about 1 hour to about 12 months.

5. The method of claim 1, further comprising causing the biological molecule to become unbound from the solid substrate.

6. The method of claim 1, wherein the biological molecule is a nucleic acid molecule.

7. The method of claim 6, wherein the nucleic acid is RNA.

8. The method of claim 1, wherein the organic solvent is ethanol, acetonitrile, acetone, tetrahydrofuran, 1,3-dioxolane, morpholine, tetraglyme, dimethyl sulfoxide, sulfolane, or a mixture of two or more of these.

9. A method for stabilizing a biological molecule bound to a solid substrate, said method comprising:
contacting the biological molecule with Tris(2-carboxyethyl)phosphine (TCEP); and
contacting the biological molecule with at least one organic solvents,
wherein said method steps results in stabilization of the biological molecule bound to the solid substrate.

10. The method of claim 9, further comprising removing some or all of the TCEP.

11. The method of claim 9, wherein the biological molecule is not contacted with (hydroxymethyl)aminomethane (Tris) or Tris-HCl.

12. The method of claim 9, further comprising storing the biological molecule in the presence of the organic solvent.

13. The method of claim 12, wherein the biological molecule is stored for a period of time between about 1 hour to about 12 months.

14. The method of claim 9, further comprising causing the biological molecule to become unbound from the solid substrate.

15. The method of claim 9, wherein the biological molecule is a nucleic acid molecule.

16. The method of claim 15, wherein the nucleic acid is RNA.

17. The method of claim 9, wherein the organic solvent is ethanol, acetonitrile, acetone, tetrahydrofuran, 1,3-dioxolane, morpholine, tetraglyme, dimethyl sulfoxide, sulfolane, or a mixture of two or more of these.

18. The method of claim 9, wherein the TCEP is provided in a composition having a pH of about 4 to about 8.

19. The method of claim 18, wherein the composition comprises 0.01 mM to 100 mM TCEP.

20. The method of claim 9, wherein contacting with at least one organic solvent creates a composition in which the organic solvent is at a final concentration of about 50% to 100% of the liquid in the composition.

21. A composition comprising:
a biological molecule bound to a solid support;
TCEP; and
at least one organic solvent,
wherein the concentrations of TCEP and organic solvent are sufficient to allow continued binding of the biological molecule to the solid substrate and to allow stabilization of the biological molecule at temperatures above 40° C.

22. The composition of claim 21, wherein the composition comprises 0.01 mM to 100 mM TCEP and the pH of the composition is from about 4 to about 8.

23. The composition of claim 21, wherein the biological molecule is a nucleic acid.

24. The composition of claim 21, wherein the nucleic acid is RNA.

25. A stabilized RNA molecule prepared by:
binding the RNA to a solid support;
contacting the RNA with TCEP; and
contacting the RNA with at least one organic solvent.

26. The stabilized RNA of claim 25, wherein the RNA can be stored for at least 1 hour without appreciable degradation.

27. The stabilized RNA of claim 25, wherein the RNA can be stored for at least 30 minutes at a temperature greater than 0° C.

28. The stabilized RNA of claim 25, wherein the RNA is bound to the solid support.

29. The stabilized RNA of claim 25, wherein the RNA is maintained in the presence of the one or more organic solvents.

30. (canceled)

31. (canceled)
